(19) United States
(12) Patent Application Publication
(10) Pub. No.: US 2012/0156715 A1
(43) Pub. Date: Jun. 21, 2012

(54) Environmental Sentinel Biomonitor System (ESB)

(76) Inventors: Mark W. Widder, Waynesboro, PA (US); Linda Brennan, Middletown, MD (US); David Trader, Smithsburg, MD (US); Tommy R. Shedd, Middletown, MD (US); William H. van der Schalie, Frederick, MD (US)

(21) Appl. No.: 13/156,826

(22) Filed: Jun. 9, 2011

Related U.S. Application Data
(60) Provisional application No. 61/400,308, filed on Jul. 16, 2010.

(51) Int. Cl. C12Q 1/18; C12M 1/34; G01N 27/02; C12Q 1/25

(52) U.S. Cl. 435/32; 435/287.1

(57) ABSTRACT

An embodiment of the invention provides a system for toxicity identification in water. The system includes a cell maintenance device having a cooling component that maintains an internal storage area of the cell maintenance device within a temperature range of 4 degrees Celsius to 25 degrees Celsius. Fluidic biochips in the cell maintenance device include one or more testing components for receiving living poikilothermic cells and at least one water test sample. A test unit receives the fluidic biochips and monitors a response to exposure of the poikilothermic cells to the water test sample. In at least one embodiment, the test unit is able to identify the degree of toxicity of the water sample within 60 minutes of receiving the fluidic biochip.

This application claims the benefit of U.S. Provisional Patent Application No. 61/400,308 filed Jul. 16, 2010, which is hereby incorporated by reference.

### I. FIELD OF THE INVENTION

The present invention is in the field of systems and methods for environmental sentinel biomonitoring.

### II. BACKGROUND

Deployed U.S. military and civilian forces face the possibility of drinking water exposed to a wide range of toxic industrial or agricultural chemicals as a result of normal use (e.g., farm run-off), damaged infrastructures, accidental spills, or deliberate chemical contamination of water. Unfortunately, rapid detection capabilities for toxic chemicals in water are limited and may not provide sufficient warning of developing toxic hazards.

### III. SUMMARY OF THE INVENTION

An embodiment of the invention provides an Environmental Sentinel Biomonitoring (ESB) system. The ESB system provides rapid toxicity identification for a broad spectrum of chemicals in water. The ESB system detects toxicity associated with non-militarized chemicals (i.e., toxic industrial chemicals (TICs) and toxic industrial materials (TIMs)) in water.

In at least one embodiment of the invention, the ESB system monitors responses of poikilothermic cells exposed to water and provides rapid responses/warnings should toxic conditions develop. Cell-based sensors integrate biological systems with electronics monitoring, facilitating rapid response to developing toxicity in water using very small systems.

The ESB system functions as a self-contained fully automated and field transportable long term (greater than or equal to 30 days) cell maintenance and water toxicity testing device. The ESB system maintains poikilothermic cells for use without the need for on-site cell culture or the use of compressed gases for maintenance. In at least one embodiment, users only have to add the samples to prepared powdered media vials and inject samples via a syringe. The EBS system in at least one embodiment also automatically processes and analyzes the samples for toxicity without human intervention.

In at least one embodiment of the invention, the ESB maintains cellular sterility in a field portable apparatus (closed system) and powdered media vials for simplified testing. The ESB also maintains cells without compressed carbon dioxide gas and it automates cellular maintenance and testing functions in one device. In at least one embodiment, the ESB provides toxicant delivery combined with cell maintenance in a ruggedized closed-system fluidic chip which utilizes edge card design for compactness and transportability. In another embodiment, the ESB merges Electric Cell-substrate Impedance Sensing (ECIS) technology with an enzyme-based acetylcholinesterase inhibition testing technology.

The ESB can be utilized for drinking water toxicity monitoring in both fixed and mobile facilities. It can also be used by water utility plants, the pharmaceutical industry, by in vitro toxicology laboratories, and for study of basic cell functions under flow conditions, and for any cellular impedance-based technology such as electroporation and cell migration.

### IV. BRIEF DESCRIPTION OF THE DRAWINGS

The present invention is described with reference to the accompanying drawings. In the drawings, like reference numbers indicate identical or functionally similar elements.

FIG. 1 illustrates a fluidic biochip seeded with RTgill-W1 cells according to an embodiment of the invention;

FIG. 2 is a table listing military exposure guideline and human lethal concentration values for test chemicals according to an embodiment of the invention;

FIG. 3 illustrates fluidic biochips inserted into a multichip reader test unit according to an embodiment of the invention;

FIG. 4 is a graph illustrating RTgill-W1 impedance readings from long term storage evaluations according to an embodiment of the invention;

FIG. 5 illustrates a graph of the average difference in sodium pentachlorophenate (PCP) response according to an embodiment of the invention;

FIG. 6 is a graph illustrating the relationship between initial cellular impedance levels of RTgill-W1 cells on fluidic biochips and response level to PCP according to an embodiment of the invention;

FIG. 7 illustrates a system for toxicity identification in water according to an embodiment of the invention; and

FIG. 8 is a flow diagram illustrating a method for toxicity identification in water according to an embodiment of the invention.

### V. DETAILED DESCRIPTION OF THE INVENTION

Exemplary, non-limiting, embodiments of the present invention are discussed in detail below. While specific configurations are discussed to provide a clear understanding, it should be understood that the disclosed configurations are provided for illustration purposes only. A person of ordinary skill in the art will recognize that other configurations may be used without departing from the spirit and scope of the invention.

An embodiment of the invention provides an Environmental Sentinel Biomonitor (ESB) system for rapid testing of drinking water supplies for toxicity from a wide range of industrial chemicals. Rather than testing for individual chemicals, the ESB system complements analyte-specific tests by providing an overall indication of toxicity. Field drinking water threats include militarized chemical agents and toxic industrial chemicals (TICs), many of which are not produced or used in the United States.

In at least one embodiment of the invention, the toxicity sensors of the ESB system respond to toxicant concentrations exceeding 7 to 14 day Military Exposure Guideline (MEG) levels, assuming an individual water consumption rate of 15 L/day typical of arid environments. An embodiment of the invention uses an electric cell-substrate impedance sensor (ECIS), which includes fluidic biochips seeded with a fish gill epithelial cell line (e.g., RTgill-W1 (ATCC CRL 2523)) to allow monitoring of impedance levels of the cells after toxicant exposure. The RTgill-W1 cell line has improved cell viability and greatly reduced logistical requirements for maintaining the cells for field use compared to cell lines of previous approaches. The ESB system in at least one embodiment allows for long term storage of fluidic biochips without feeding with the ability to detect the same chemicals below the Human Lethal Concentration (HLC), for example, at two weeks and one year of storage.

In at least one embodiment of the invention, rainbow trout cells, more particularly rainbow trout gill epithelial cells (RTgill-W1), are used as the biological component of a cell-based biosensor for detection of the presence of environmental toxic industrial chemicals in water supplies. In an alternate embodiment, the RTgill-W1 cells may be replaced by other cold blooded animal cells. RTgill-W1 cells seeded on enclosed fluidic biochips and monitored with ECIS technology can detect 18 out of the 18 TICs tested within one hour; nine of these within the specified concentration range between the MEG concentration and the HLC. In at least one embodiment, the RTgill-W1 cells are stored on the biochips at ambient carbon dioxide levels at 4-6 degrees Celsius (° C.) for over one year without feeding or media changes.

The ESB system uses the measurement of electrical impedance of cell monolayers as a toxicity sensor (to measure the degree of toxicity), which is also known as ECIS. Cellular impedance is a sensitive indicator of cell viability and cytotoxicity. ECIS is based on the principal that a confluent monolayer of cells impedes the flow of electricity. Impedance measurements correlate with changes in cellular morphology, movements and functions, and activation of signaling pathways. When the integrity of the cell monolayer is compromised (as by a toxic chemical injury), the ECIS sensor records a change in electrical impedance.

RTgill-W1 cells form a monolayer on ECIS fluidic biochips, can be cultured at ambient CO₂ levels, and can survive over a wide range of temperatures (4° C. to 25° C.), with optimal growth occurring at 20±2° C. RTgill-W1 cells are sensitive to different classes of compounds, including polybrominated diphenyls, ammonia, heavy metals such as copper, polycyclic aromatic hydrocarbons, industrial effluents, and nanoparticles.

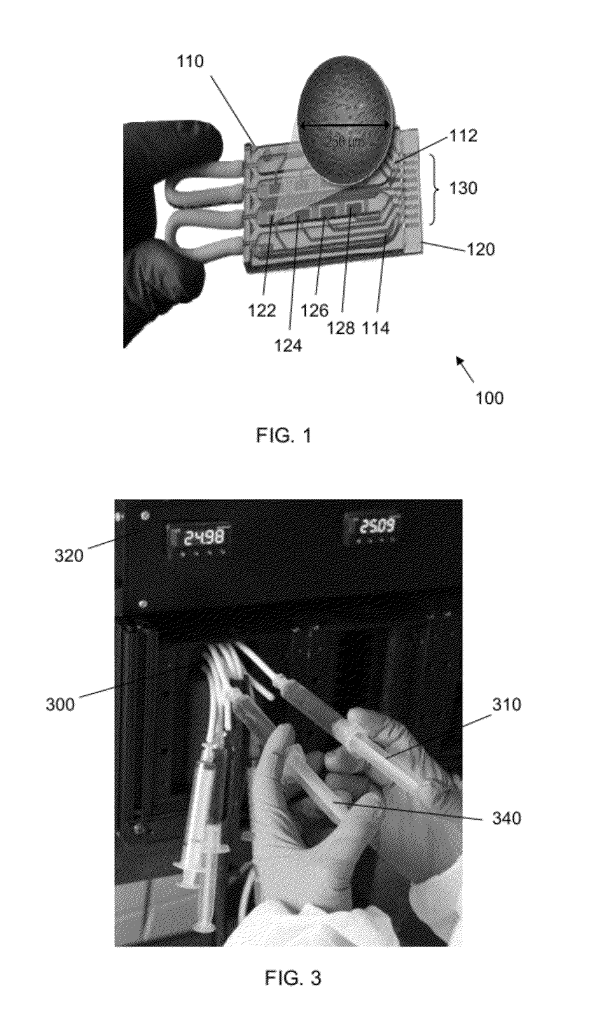

The following description provides an example method to seed a fluidic biochip with RTgill-W1 cells (FIG. 1) according to an embodiment of the invention. Seeded biochips 100 are sealed and are able to be stored for up to one year without feeding and maintenance. Sterile fluidic biochips (available from Agave BioSystems, Austin, Tex.) are assembled from two components: an upper polycarbonate layer 110 with two separate fluid channels 112 and 114, and a lower electronic layer 120 containing four electrode pads 122, 124, 126, and 128 per channel for impedance sensing. There are 10 working electrodes per pad that are each 250 μm in diameter (an example is illustrated by the enlarged section in FIG. 1). The assembled biochips have electrode connections 130 (for example made from gold) for acquiring impedance readings when inserted into the ECIS test unit, which is described below.

RTgill-W cells (available from American Type Tissue Culture Collection, Manassas, Va.) are a continuous immortalized cell line. The cells are cultured in 75 cm² polystyrene flasks in complete Leibovitz-15 (L-15) media containing 1% 200 mM GlutaMAX-1 supplement (v/v), and 100 U/mL penicillin and 100 U/mL streptomycin (culture media ingredients available from Lonza, Walkersville, Md.) in a 20° C. incubator with ambient carbon dioxide (CO₂). The cells are used at passages 5-55 for seeding the fluidic biochips.

Prior to being seeded with cells, the channels of the fluidic biochips are pre-coated with 0.01% fibronectin solution (available from Calbiochem, Gibbstown, N.J.) in L-15 media for 1 hour in order to facilitate cell attachment and the formation of a continuous epithelial monolayer. After 1 hour of incubation at room temperature, the fibronectin solution is aspirated off and the biochips are seeded with 2.5 mL of 2.5×10⁵ cells/mL of trypsinized cells per channel. Sterile Pharmed® Biopharm tubing (available from McMaster-Carr, Santa Fe Springs, Calif.) is used to form closed loops on the ends of the chips (FIG. 1). Bare electrodes that contain culture media and no cells have impedance values of 300-400 ohms (Ω). Once the RTgill-W1 cells are introduced to the fluidic biochip and the monolayer is formed, impedance values range from approximately 1200-2000Ω. Seeded chips are incubated at 20° C. for 7 days. During this time, the media in the fluidic channels is replenished on days 4 and 7 prior to storing the chips at 6° C. on day 7. A set of seeded chips (11 days to 18 days post-seeding) is used for chemical testing; another set is used for long-term storage testing.

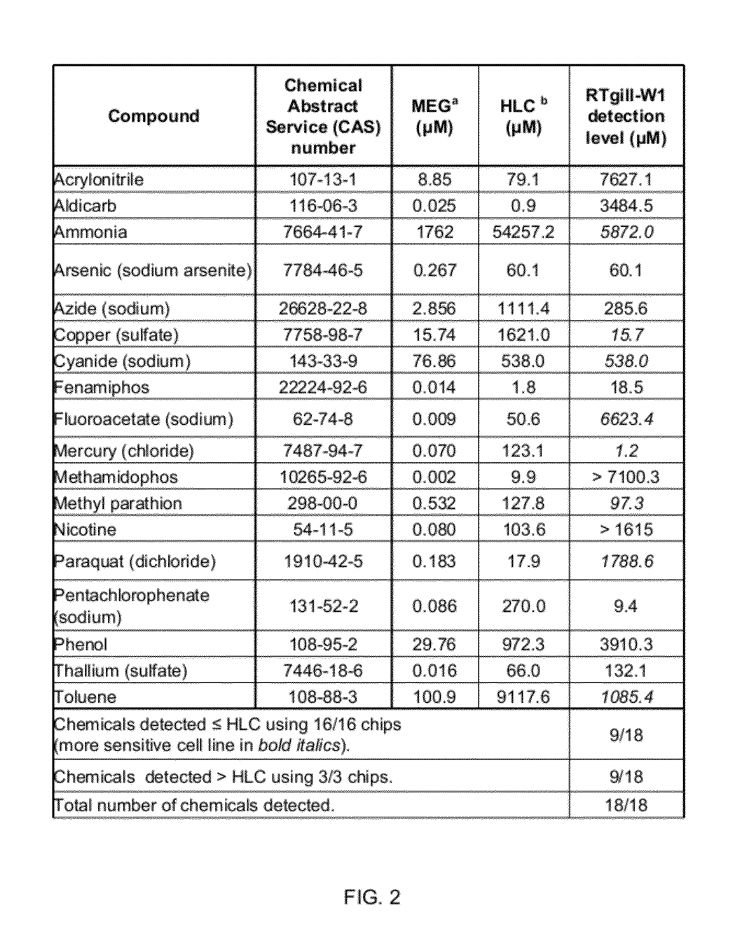

The following description provides an example of a method of preparing and analyzing test chemicals according to an embodiment of the invention. Eighteen TICs are evaluated for ECIS toxicity with the fluidic biochips. Desirable concentration ranges for toxicity detection for the TICs are determined to be above the Military Exposure Guideline (MEG) concentration (based on consumption of 15 L per day of water for 7-14 days) and below the estimated Human Lethal Concentration (HLC; based on the consumption of 15 L of water per day for a 70 kilogram person). FIG. 2 is a table listing the MEG and HLC values for the test chemicals. Test chemicals include: acrylonitrile, aldicarb, sodium arsenite, fenamiphos, methamidophos, methyl parathion, nicotine, paraquat dichloride (available from Chem Service, West Chester, Pa.), ammonium chloride, copper sulfate, sodium cyanide, sodium fluoroacetate, mercuric chloride, phenol, thallium sulfate, and toluene (available from Sigma-Aldrich, St. Louis, Mo.), and sodium pentachlorophenate (PCP) (prepared from pentachlorophenol, which is available from Mallinckrodt Baker, Phillipsburg, N.J.).

Test compound stock solutions are prepared in deionized water (DI) with the exception of PCP, which is prepared from pentachlorophenol in 5 mM phosphate buffer, pH adjusted to 7.5. Stock concentrations of test compounds are analyzed using the following analytical methods: acrylonitrile, aldicarb, fluoroacetate, methyl parathion, nicotine, paraquat, pentachlorophenol, and phenol are analyzed by high performance liquid chromatography (HPLC). Ammonia is measured colorimetrically using a LaMotte 1200 Colorimeter. Arsenic, copper, mercury, and thallium are measured by inductively coupled plasma-mass spectrophotometry (ICP-MS). Fenamiphos and toluene are measured with a gas chromatograph (GC). An ion probe is used to measure cyanide. Azide is measured using ion chromatography. Methamidophos is tested at nominal concentrations. Volatile chemicals (acrylonitrile and toluene) are stored in zero headspace vials at 4° C. All test chemicals are stable for two weeks. On the day of ECIS testing, stock solutions of the selected compounds are diluted with DI water to obtain desired concentrations.

The following description provides an example of a method for ECIS chemical testing with RTgill-W1 fluidic biochips according to an embodiment of the invention. The toxicant testing of the fluidic biochips is based upon a statistical approach for determining a minimum detection limit (MDL) for each test chemical. The MDL is the lowest tested concentration at which there is a 90% probability of detection with 80% confidence. Based on binomial probabilities, a minimum of 16 of 16 samples must be detected with no false negatives. Each fluidic chip has 2 channels for testing; one channel is used for a control, and the other for the test compound. ECIS testing for the RTgill-W1 cells is normally performed at 25° C. but can be tested at other temperatures, for example, between 4 and 25 degrees Celsius as long as both the control and treatment solutions are equilibrated to the same temperature. L-15ex powdered media with phenol red (available from US Biological, Swampscott, Mass.) is used as the test media for both the control and treatment injections. L-15ex is a modified version of L-15 media and contains the same concentrations of salts, galactose, and pyruvate as basal L-15, but no vitamins or amino acids. A pre-exposure period is initiated by injecting each channel of the biochip 300 with 10 mL of L-15ex control media over a one minute period using a 10 mL syringe 310 as illustrated, for example, in FIG. 3. Since the media environment surrounding the monolayers of RTgill-W1 cells within the chips is being changed within the chips from complete growth media to test media, this pre-exposure period allows the impedance levels of the cells to reach equilibrium before introducing the test compound. Each chip is then inserted into the test unit 320, and the ECIS software is started to collect 30 minutes of pre-exposure data with one minute readings immediately after the injections.

During the pre-exposure period, desired concentrations and volumes of the test chemical are prepared in powdered L-15ex media along with replicate control solutions and equilibrated to 25° C. or other desired temperature. Inclusion of the pH indicator phenol red in the media facilitates visual comparisons between the color of the control and the test samples. pH differences that are greater than 0.20 pH units cause changes in media color that can be detected visually. In these instances, test samples are adjusted with 20% HCl or 1.0 N NaOH until the pH of the test sample is the same visually as the control sample to prevent pH-related ECIS responses. At the end of the minute pre-exposure, one channel of each of the biochips is injected with 10 mL of control media (syringe 310 in FIG. 3), and the other channel is injected with 10 mL of test media solution (syringe 340 in FIG. 3). This is done while the chips are in place in the test unit and impedance data is still being collected as illustrated, for example, in FIG. 3. FIG. 3 illustrates multiple fluidic biochips inserted into a multichip reader test unit for 1 hour exposure testing according to an embodiment of the invention. The figure depicts a manual exposure injection with attached syringes of control and test samples in L15ex media. The ECIS software continuously collects the average raw impedance values from each of the four electrode pads in each channel, for example, every 60 seconds although other epoch lengths could be used. The data is normalized and displayed on the ECIS reader monitor as a real-time graph. There is a range of starting impedance values on each electrode due to variability of the cell layers covering the electrodes. Normalizing the data allows for a more uniform comparison of the impedance values between the electrodes, and subsequently, between experiments. The normalized impedance values at each time point is calculated by dividing by the initial impedance values just prior to the exposure period on each electrode. After one, four, and eight hours, the impedance data is analyzed using curve discrimination software to compare differences between the control channels and the treated channels (see statistical analysis below).

The following description provides an example of a method for long-term testing of RTgill-W1 fluidic biochips according to an embodiment of the invention. In order to study the effects of cold storage with no media replenishment on the viability and sensitivity of the RTgill-W1 cells, fluidic biochips are seeded, held at 20° C., and media changes are performed on days 4 and 7 as described above, then held at 6° C. with no media replenishment for up to one year. Three chips are removed for viability assessment and ECIS testing using the 9.4 μM detection level for PCP (FIG. 1) at 4, 8, 12, 16, 20, 24, 32, 38, and 52 weeks and the chemical exposure procedure described above.

To determine the length of cell viability at temperatures above 6° C., 9 fluidic biochips seeded with RTgill-W1 cells are placed in a thermoelectric ECIS unit where impedance values are recorded every hour for up to 5 months. Three chips each are held at 12° C., 20° C. and 25° C. Viability of the cell monolayer is determined by impedance readings over the specified time period.

The following description provides an example of a method for statistical analysis according to an embodiment of the invention. A curve discrimination program using MATLAB (available from The MathWorks, Inc., Natick, Mass.) is used to perform the statistical analysis. The program is used to determine when the response curves generated by toxicant-exposed cells differ significantly from the curves generated by the controls. This program analyzes impedance data from control and toxicant-exposed cells every minute during a 60 minute window. The overall difference between the two groups of curves is compared at each time point, and the difference between their averages is assessed relative to the within-group variability. A confidence level of 99% (p<0.001) is used to establish statistical significance. Functional data analysis techniques are used to extend the standard single time point analysis of variance approach to a curve consisting of approximately 60 points. The lowest toxicant concentrations that cause a significant change in impedance in at least ten consecutive time intervals over a 60 minute period are reported as the detection levels in FIG. 1.

The table in FIG. 2 compares and summarizes the ECIS toxicity test results of the RTgill-W1 seeded fluidic biochips. With the RTgill-W1 chips, 9 out of the 18 test chemicals are detected after a one hour exposure at concentrations that are between the MEG and the HLC. These chemicals are ammonia, arsenic, azide, copper, cyanide, mercury, methyl parathion, pentachlorophenate, and toluene. All of these compounds cause a significant decrease (p<0.001) in impedance compared to controls, with the exception of copper, which causes an increased impedance compared to the controls. Acrylonitrile, aldicarb, fenamiphos, fluoroacetate, methamidophos, nicotine, paraquat, phenol, and thallium are detected in 3 out of 3 chips, but at levels above the HLC.

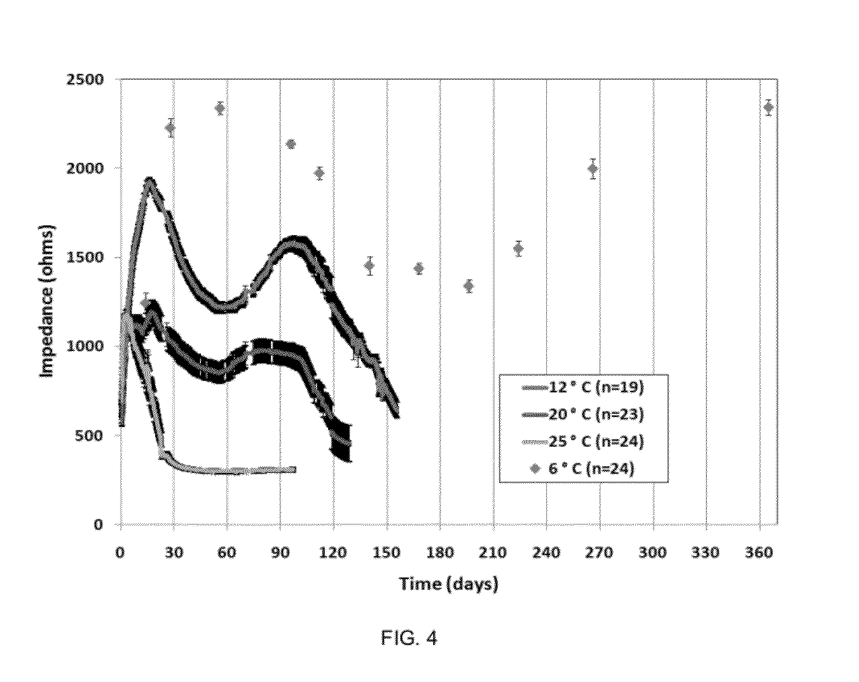

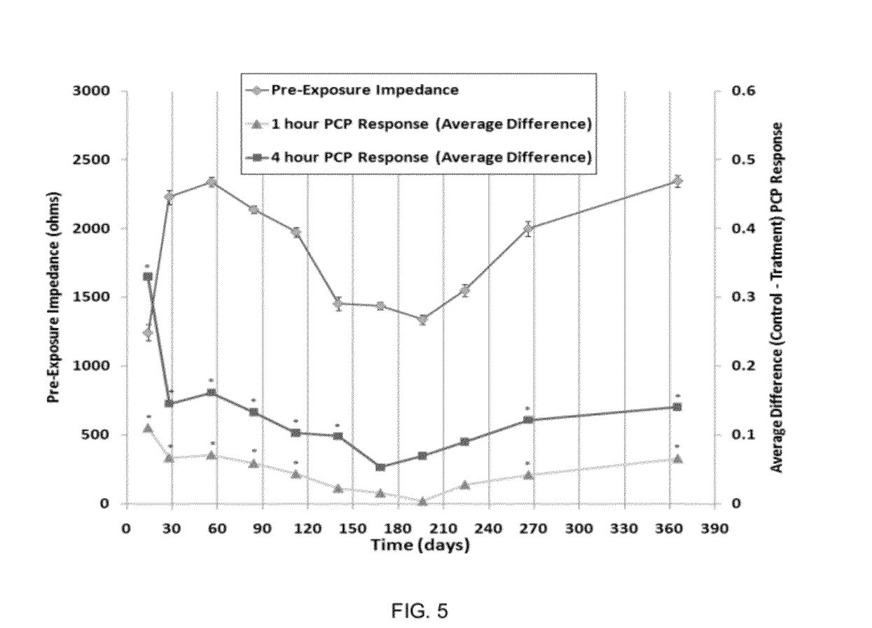

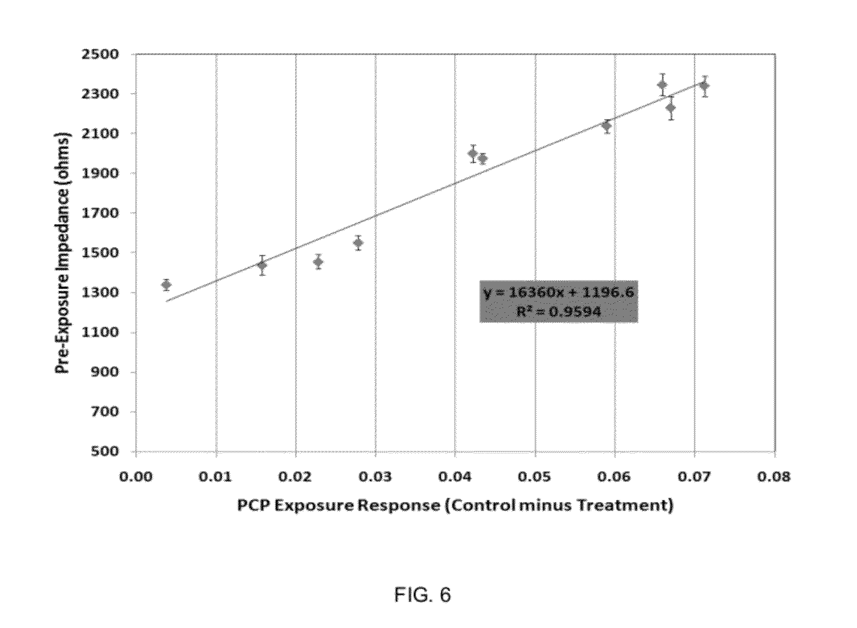

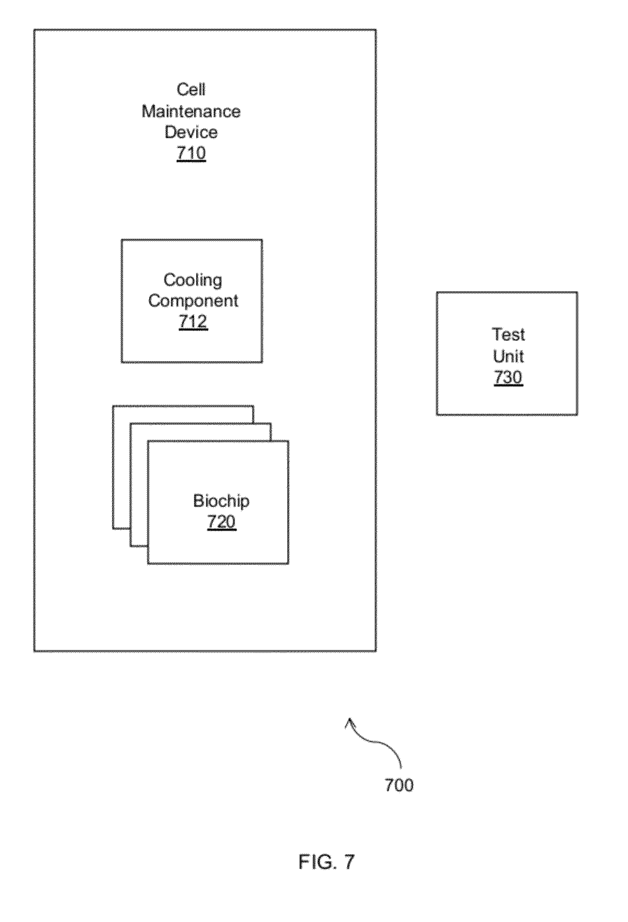

An embodiment of the invention optimizes storage conditions for a monolayer of cells on a fluidic biochip to produce a field-portable biosensor that requires little or no maintenance. The RTgill-W1 cell line can withstand long-term storage in refrigerated temperatures. The physiology of a poikilothermic organism, such as the rainbow trout, allows these organisms to survive at relatively low ambient temperatures in vivo. FIG. 4 illustrates impedance data and demonstrates that the RTgill-W1 cells can be maintained at reduced temperatures for extended periods of time without feeding or maintenance. FIG. 4 is a graph illustrating RTgill-W1 impedance readings from long term storage evaluations according to an embodiment of the invention. The 12° C., 20° C. and 25° C. data are from the study described above and represent continuous hourly impedance readings from replicate fluidic biochips at the different temperatures. The 6° C. data represents a separate batch of fluidic biochips that were seeded in a separate study. Triplicate chips are evaluated from this batch of chips at various time points during a one year time span and tested against a known toxicant (toxicity results are shown in FIGS. 6 and 7). Error bars in FIG. 6 represent standard error of mean for the replicate chips. The cells were not fed during the monitoring period. There was an increase in the shelf-life of the seeded chips with decreased temperatures. As described above, fluidic biochips that contain media and no cells have raw impedance values ranging from 300-400Ω. Once a cell monolayer is established on the chips, the impedance values for RTgill-W1 cells range from 1200-2000Ω when stored at 20° C. As can be seen in FIGS. 5 and 6, these impedance levels increase to about 2200Ω and 2400Ω at 30 and 60 days, respectively, when stored at 6° C. At 20° C., the biochips maintain a viable monolayer of cells for over 3 months without media renewal. Decreasing the storage temperature to values below the recommended culture temperature of 20° C. provides two distinct advantages for long-term biosensor development. First, the shelf-life of the biochips increases to over 4 months when held at 12° C., and to over one year when held at 6° C. Second, a considerable increase in monolayer impedance is observed at the 12° C. and 6° C. storage temperatures as compared to biochips held at 20° C. and 25° C. FIG. 4 also shows that storage temperatures above 20° C. lead to a decrease in shelf life; with less than 30 days at 25° C. At 30° C., fluidic biochips are viable for less than 24 hours.

Fish can withstand relatively long periods of starvation without severe consequences, and water temperature influences long-term fish survival by altering their metabolism. Rainbow trout, Oncorhynchus mykiss, can survive in waters between 0 and 29.8° C. dependent upon temperature adaptation and fish strain. RTgill-W1 cells are able to survive for over a year at 6° C. on a biochip as discussed above.

FIG. 5 shows that the biochips held at 6° C. maintain high impedances even after 12 months of storage. Unfed RTgill-W1 cells are maintained at 6° C. for different durations and subsequently tested with PCP exposures at 9.4 μM. The line with diamond data points shows the initial impedance prior to PCP exposure. The line with triangle data points and the line with square data points show, respectively, 1-hour and 4-hour average of response difference between the control and treatment channels at each respective storage time for 3 replicate fluidic biochips. Significance at each time point (p<0.001) is depicted by an * and requires a positive detection for all 3 replicate chips using the curve discrimination program. The replication of these impedance level patterns at all three storage temperatures indicates that the effect may be linked to the metabolic machinery of the cells, which is reduced at lower temperatures. If the impedance patterns shown for the 12° C. and 20° C. chips are predictive indicators of the impedance patterns of the 6° C. chips, the 6° C. chips may remain viable for approximately 18 months, but most likely not beyond 2 years of storage.

FIG. 5 shows the toxicity results of fluidic biochips in relationship to the impedance levels of the chips that are stored at 6° C. The graph displays pre-exposure initial impedance data (diamonds) along with the average normalized impedance difference (n=3 chips per time point) between the treatment and control response channels of the fluidic biochips after exposure to 9.4 μM of PCP for 1 hour and for 4 hours. There is a positive response to PCP exposure at all the time points indicated, and there is a strong relationship between the impedance levels and the magnitude of PCP response except at the first two week time period, which shows the greatest difference between treatment and control channels. As can be seen in FIG. 5, the marked decreases in the initial impedance values at 140 through 224 days at 6° C. storage correlates with decreased responses of the biochips to the toxicant. When the exposure period evaluated is for 1 hour, responses are significant at time points of 14-114 days, and also at 364 days. At a PCP exposure time of 4 hours, additional significant responses are obtained at 140 and 266 days. Evaluation of the PCP exposures after 8 hours does not result in an improvement of the responsiveness of the biochips as compared to 4 hours. For both the 1 hour and 4 hour PCP exposures, the biochips are significantly responsive after one year storage at 6° C.

FIG. 6 is a graph illustrating RTgill-W1 fluidic biochip exposures to 9.4 μM PCP for one hour according to an embodiment of the invention. The x-axis represents the difference between the average control and treatment impedance for fluidic biochips. Individual data points are averaged by length of storage. The y-axis depicts the average impedance prior to toxicant exposure for each storage time point. The data is plotted independent of storage time and thus a strong correlation between initial impedance and exposure response strength can be seen. FIG. 6 shows a strong positive linear relationship between the pre-exposure impedance level and the magnitude of response to a one hour exposure of 9.4 μM PCP (R²=0.96) for chips evaluated from 1 month to 12 months of storage. This relationship is independent of storage time because as the impedance levels increase, the toxicant sensitivity of the chips also increases. Chips that are stored for only 2 weeks do not fit this model as they have considerably higher response levels to PCP exposure. While a decreasing sensitivity trend is apparent through the first six months of the storage period, the second half of the storage period shows a trend of increasing toxicant sensitivity to PCP through one year (FIG. 5). This increase in toxicant sensitivity is related to the increased cellular impedance observed in the later storage times. Accordingly, the fluidic biochips seeded with RTgill-W1 cells are able to maintain a confluent monolayer for at least 1 year when stored at 6° C. without media replenishment and are still responsive to the benchmark toxicant, PCP, when used in the ECIS assay. The RTgill-W1 biochips are suitable in field-portable biosensors for rapid toxicity detection of chemicals in drinking water supplies because of their sensitivity to chemicals combined with their long-term storage capabilities.

At least one embodiment of the invention provides a method for maintaining and testing animal cell lines without the need for routine feeding. Cell types derived from poikilothermic animals such as fish and frogs have been found to withstand colder temperatures than mammalian cell lines. Through means of refrigeration, cells can be maintained on the chips in a confluent state with high electrical impedances for many months without feeding. Whenever chips are needed for testing a water sample, the chips are removed from refrigeration and are ready for testing. The following description details the function of the ESB system with one specific cell type according to an embodiment of the invention. In other embodiments, other poikilothermic cell types can be used within the same platform.

Example 1 materials utilized include: fibronectin, L-15ex powdered media with phenol red; (available from US Biological, Swampscott, Mass.); rainbow trout gill W-1 cells (RTgill-W1) (available from American Type Tissue Culture Collection, Manassas, Va.); 60 mil lexan fluidic biochips (available from Applied BioPhysics, Troy, N.Y.); 10 and 20 mL syringes with BD Luer-Lok Tip; (available from VWR Scientific, West Chester, Pa.); Tygon® Pharmed® tubing; (available from United Plastic Corp, Lima, Ohio); female Luer thread style barbs; (available from Value, Plastics, Fort Collins, Colo.); and press-in plugs (available from Value Plastics). Other materials include L-15 media; GlutaMAX-1 Supplement; and, Penn/Strep stock; 10K/10K (all available from Lonza, Walkersville, Md.).

To prepare a fibronectin substrate, 1 mg of fibronectin is diluted in 100 mL of L-15 media to be used as a substrate for the biofluidic chips at a concentration of 10 ug/mL. This solution can be stored in 10 mL aliquots at −20° C. RTgill W-1 complete growth media is prepared by adding 50 mL of FBS, 5 mL of 200 mM GlutaMAX-1 Supplement, and 5 mL of Penn/Strep stock to 500 mL of L-15 and refrigerating. To prepare RTgill-W1 cells for seeding of biofluidic chips, RTgill W-1 cells are harvested on the day of chip seeding and diluted to a concentration of 2.5×10⁵ cells/mL in RTgill W-1 complete growth media.

The seeding of biofluidic chips in this example was carried out in a sterile biohazard hood using an aseptic technique. More specifically, the biofluidic chips are logged in the date of receipt and given a unique number for chip tracking purposes. There are 2 channels on each chip that receive 2.5 mL per channel of 2.5×10⁵ cells/mL. One confluent T75 flask yields approximately 1×10⁷ RTgill W-1 cells. Therefore, one confluent T75 flask can seed 8 chips. The number of chips to be seeded each week depends on the number of chemicals to be run.

Sixty minutes prior to seeding the chips, 2.5 mL of the 10 ug/mL fibronectin solution are injected into each channel of the chip. The chips are left in the sterile biohazard hood for 60 minutes, and then flushed with sterile serum-free L-15 media before seeding with cells. Two 7 cm sections of autoclaved tubing are placed on the inside hose barbs of the chips. The calculated number of confluent RTgill W-1 flasks is trypsinized and the cell suspensions are combined in a sterile 300 mL plastic container. A 10 μL aliquot of the cells are counted using a hemacytometer, and the volume of complete RTgill W-1 growth media needed to attain a cell suspension of 2.5×10⁵ cells/mL is calculated.

Using a sterile 20 mL syringe, 2.5 mL of the cell suspension are injected into each channel of the chip that does not contain the hose pieces. The tube over the other hose barb is closed for each channel. The chips are placed in large sterile plastic Petri dishes (6 chips per dish), which are placed in a 20° C. incubator. On day 8, the chips are removed from the 20° C. incubator, fed with 2.5 mL per channel of RTgill W-1 complete growth media in the sterile biohazard hood, and placed in a 6° C. refrigerator. The chips may be stored without feeding until needed for testing.

To perform chemical testing with the seeded ECIS chips, the test sample or deionized control water is reconstituted in separate vials of premeasured powdered L15-EX media as needed. These test solutions are placed in a 25° C. incubator for 30 minutes prior to use. The chips are removed from the 6° C. refrigerator. Each chip has 2 channels: one to be used for a control injection, and one to be used for the test sample. From this point on, the chips no longer need to be handled under sterile conditions. The chips are removed from the Petri dishes, and the hose loops are opened so that the hoses are on the inner barbs. These hoses will serve as the drains. Two 7 mm sections of hose with female Luer thread style barbs are placed on the ends on the outside hose barbs of the chips.

10 mL syringes are filled with pre-warmed L-15ex media and the syringes are locked onto luer locks on the chips. Each channel of each chip is hand-injected and a plug is placed on the end of each outlet hose after injection. The syringes are left in place on the chips. Each chip is placed in the ECIS test unit in designated slots. 30 minute pre-exposure data is collected using the ECIS curve discrimination program. Each data set is automatically stamped with the date, time and test unit position number of the chip.

After 20 minutes of pre-exposure, 10 mL syringes are filled with either control or test chemical media that has been incubating at 25° C. The empty pre-exposure syringes are systematically removed from the test chips and replaced with the appropriate control (channel A) or test chemical (channel B) syringes. When the 30 minute pre-exposure period is finished, the chips are manually injected with appropriate test compounds and control media with approximately a 30 second injection. Impedance values are collected, for example, for another hour, two hours, eight hours, or overnight.

FIG. 7 illustrates a system 700 for toxicity identification in water according to an embodiment of the invention. The system includes a cell maintenance device 710 having a cooling component 712. In at least one embodiment, the cooling component 712 (e.g., refrigeration unit) maintains an internal storage area of the cell maintenance device 710 within a temperature range of 4° C. to 25° C. The cell maintenance device 710 is a portable apparatus for storing and maintaining (i.e., keeping alive) fluidic biochips 720 that can be used for field testing water samples.

In at least one embodiment of the invention, the fluidic biochips 720 include one or more testing components (also referred to herein as “channels”) for receiving living poikilothermic cells and a water test sample. As described more fully above, the poikilothermic cells are seeded within the fluidic biochips 720 before exposure to water test samples. In at least one embodiment, the poikilothermic cells include fish cells (e.g., rainbow trout gill epithelial cells) or frog cells.

In at least one embodiment of the invention, the poikilothermic cells include an eel (e.g., American eel) or fathead minnow cell line. In another embodiment, the ECIS toxicant sensitivity response is increased by using vertebrate cell lines with engineered receptors, and by using multiparameter biochips that measure pH, oxygen, and optical changes in cells as well as ECIS. In yet another embodiment, enzymes, bacteria, and/or yeast that can complement ECIS response capabilities are used in the biosensors. In at least one embodiment, enzymatic components are added to pick up the acetylcholinesterase inhibiting chemicals. As also described more fully above, the cell maintenance device 710 allows for long term storage of fluidic biochips 720 having viable poikilothermic cells without feeding and maintenance with little or no loss in toxicant sensitivity.

The system 700 further includes a test unit 730 for receiving the fluidic biochips 720. The test unit 730 monitors a response to exposure of the poikilothermic cells to the water test sample. In at least one embodiment, the test unit 730 includes a cell-based electrical impedance sensor. In another embodiment, the test unit 730 includes an enzyme-based sensor. In yet another embodiment, the cell maintenance unit 710 comprises the test unit 730.

In at least one embodiment of the invention, the test unit 730 is able to detect a toxicant in the water sample within 60 minutes of receiving the fluidic biochip 720. More specifically, cellular biosensors measure changes in cell behavior, which correlates well with toxicity. In at least one embodiment, the toxicants include. acrylonitrile, aldicarb, sodium arsenite, azide, fenamiphos, methamidophos, methyl parathion, nicotine, paraquat dichloride, ammonium chloride, copper sulfate, sodium cyanide, sodium fluoroacetate, mercuric chloride, phenol, thallium sulfate, toluene, and/or sodium pentachlorophenate.

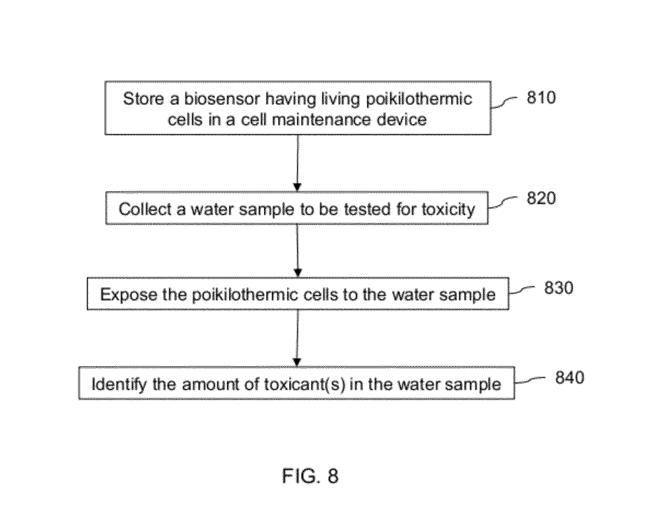

FIG. 8 is a flow diagram illustrating a method for toxicity identification in water according to an embodiment of the invention. A biosensor having living poikilothermic cells is stored in a temperature-controlled (e.g., refrigerated) cell maintenance device 810. In at least one embodiment, the cell maintenance device includes a cooling component that maintains the biosensor at a temperature range of 4° C. to 25° C.

A water sample to be tested for toxicity is collected 820; and, the poikilothermic cells in the biosensor are exposed to the water sample 830. As described more fully above, two channels in the biosensor are pre-seeded with living poikilothermic cells (e.g., fish or frog cells) prior to insertion of a test water sample and a control water sample. The biosensor includes a fluidic biochip, a cell-based electrical impedance sensor, and/or an enzyme-based sensor.

The biosensor identifies the degree of toxicity in the water sample 840. In at least one embodiment, the toxicants include acrylonitrile, aldicarb, sodium arsenite, azide, fenamiphos, methamidophos, methyl parathion, nicotine, paraquat dichloride, ammonium chloride, copper sulfate, sodium cyanide, sodium fluoroacetate, mercuric chloride, phenol, thallium sulfate, toluene, and/or sodium pentachlorophenate. In at least one embodiment, of the invention, the degree of toxicity in the water sample is identified within 60 minutes of exposure of the poikilothermic cells to the water sample.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means plus function elements in the claims below are intended to include any structure, or material, for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A system for toxicity identification in water, said system comprising:
a cell maintenance device comprising a cooling component;
at least one fluidic biochip in said cell maintenance device, said fluidic biochip having a testing component for receiving living fish cells and at least one water test sample; and
a test unit for receiving said fluidic biochip, said test unit monitors a response to exposure of said living fish cells to the water test sample.

2. The system according to claim 1, wherein said fish cells include rainbow trout gill epithelial cells.

3. The system according to claim 1, wherein said test unit include a cell-based electrical impedance sensor.

4. The system according to claim 1, wherein said test unit includes an enzyme-based sensor.

5. The system according to claim 1, wherein said cooling component maintains an internal storage area of said cell maintenance device within a temperature range of 4° C. to 6° C.

6. The system according to claim 1, wherein said test unit identifies a degree of toxicity in the water sample within 60 minutes of receiving said fluidic biochip.

7. The system according to claim 6, wherein the toxicant includes at least one of acrylonitrile, aldicarb, sodium arsenite, azide, fenamiphos, methamidophos, methyl parathion, nicotine, paraquat dichloride, ammonium chloride, copper sulfate, sodium cyanide, sodium fluoroacetate, mercuric chloride, phenol, thallium sulfate, toluene, and sodium pentachlorophenate.

8. A system for toxicity identification in water, said system comprising:
a cell maintenance device comprising a cooling component said cooling component maintains an internal storage area of said cell maintenance device within a temperature range of 4° C. to 25° C.;
at least one fluidic biochip in said cell maintenance device, said fluidic biochip having a testing component for receiving living poikilothermic cells and a water test sample; and
a test unit for receiving said fluidic biochip, said test unit monitors a response to exposure of said living poikilothermic cells to the water test sample.

9. The system according to claim 8, wherein said living poikilothermic cells include at least one of fish cells and frog cells.

10. The system according to claim 9, wherein said fish cells include rainbow trout gill epithelial cells.

11. The system according to claim 8, wherein said test unit include a cell-based electrical impedance sensor.

12. The system according to claim 8, wherein said test unit includes an enzyme-based sensor.

13. The system according to claim 8, wherein said test unit identifies an amount of at least one toxicant in the water sample within 60 minutes of receiving said fluidic biochip.

14. The system according to claim 13, wherein the toxicant includes at least one of acrylonitrile, aldicarb, sodium arsenite, azide, fenamiphos, methamidophos, methyl parathion, nicotine, paraquat dichloride, ammonium chloride, copper sulfate, sodium cyanide, sodium fluoroacetate, mercuric chloride, phenol, thallium sulfate, toluene, and sodium pentachlorophenate.

15. A method for toxicity identification in water, said method comprising:
storing a biosensor in a temperature-controlled cell maintenance device, the biosensor including living poikilothermic cells;
collecting a water sample to be tested for toxicity;
exposing the living poikilothermic cells in the biosensor to the water sample; and
detecting a change in impedance as a representation of a degree of toxicity in the water sample.

16. The method according to claim 15, wherein the living poikilothermic cells include at least one of fish cells and frog cells.

17. The method according to claim 15, wherein the biosensor includes one of a fluidic biochip, a cell-based electrical impedance sensor, and an enzyme-based sensor.

18. The method according to claim 15, wherein said storing of the biosensor includes storing the biosensor in the cell maintenance device at a temperature range of 4° C. to 25° C.

19. The method according to claim 15, wherein said detecting of the change in impedance comprising identifying a degree of toxicity in the water sample within 60 minutes of said exposing of the living poikilothermic cells to the water sample.

20. The method according to claim 19, wherein the toxicant includes at least one of acrylonitrile, aldicarb, sodium arsenite, azide, fenamiphos, methamidophos, methyl parathion, nicotine, paraquat dichloride, ammonium chloride, copper sulfate, sodium cyanide, sodium fluoroacetate, mercuric chloride, phenol, thallium sulfate, toluene, and sodium pentachlorophenate.
